(19) United States
(12) Patent Application Publication
(10) Pub. No.: US 2009/0149495 A1
(43) Pub. Date: Jun. 11, 2009

(54) COMPOUNDS

(76) Inventors: Katalin Nogradi, Budapest (HU); Gabor Wagner, Budapest (HU); Gyorgy Keseru, Telki (HU); Attila Bielik, Orkeny (HU); Tamas Gati, Budapest (HU); Viktor Hada, Budapest (HU); Janos Koti, Tiszavasvari (HU); Krisztina Gal, Budapest (HU); Monika Vastag, Budapest (HU); Amrita Agnes Bobok, Budapest (HU)

Correspondence Address: FISH & RICHARDSON P.C., PO BOX 1022, MINNEAPOLIS, MN, 55440-1022, US

(21) Appl. No.: 12/158,580

(22) PCT Filed: Dec. 19, 2006
(86) PCT No.: PCT/HU2006/000122

§ 371 Date: Nov. 4, 2008

(30) Foreign Application Priority Data
Dec. 20, 2005 (HU) P05 01166
Dec. 18, 2006 (HU) P06 00919

(51) Int. Cl. A61K 31/4365; C07D 471/04; A61P 25/00

(52) U.S. Cl. 514/301; 546/114

(57) ABSTRACT

The present invention relates to new mGluR1 and mGluR5 receptor subtype preferring ligands of formula (I) wherein X represents a group selected from CO, SO, SO₂; Y represents a group selected from 0, OCH₂, (CH₂)n, NH, NHCH₂; n is an integer of 0 to 2; R₁ is an optionally substituted alkyl, cycloalkyl, phenyl, biphenyl, heterocyclyl; R₂ is an optionally substituted phenyl, heterocyclyl or NR₃R₄ group wherein R₃ and R₄ are independently selected from the group of hydrogen, alkyl, or R₃ and R₄ together with the N atom to which they are attached can form an optionally substituted C₅₋₇ heterocyclyl group, containing one or more heteroatom(s) selected from the group of N, O, S, and/or tautomers and/or salts and/or hydrates and/or solvates thereof, to the processes for producing the same, to pharmaceutical compositions containing the same and to their use in therapy and/or prevention of pathological conditions which require the modulation of mGluR1 and mGluR5 receptors such as neurological disorders, psychiatric disorders, acute and chronic pain, neuromuscular dysfunctions of the lower urinary tract and gastrointestinal disorders.

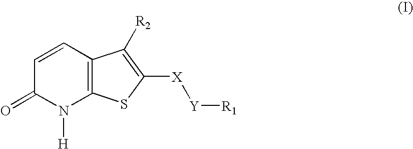

### FIELD OF THE INVENTION

The present invention relates to new mGluR1 and mGluR5 receptor subtype preferring ligands of formula (I) and/or tautomers and/or salts and/or hydrates and/or solvates thereof, to the processes for their preparation, to pharmaceutical compositions containing these compounds and to their use in therapy and/or prevention of a condition which requires modulation of mGluR1 and mGluR5 receptors.

### BACKGROUND OF THE INVENTION

A major excitatory neurotransmitter in the mammalian central nervous system (CNS) is the glutamate molecule, which binds to neurons, thereby activating cell surface receptors. These receptors can be divided into two major classes, ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A2; increases in arachidonic acid release; and increases or decreases in the activity of voltage- and ligand-gated ion channels. (Trends Pharmacol. Sci., 1993, 14, 13; Neurochem. Int., 1994, 24, 439; Neuropharmacology, 1995, 34, 1; Prog. Neurobiol., 1999, 59, 55; Berl. Psychopharmacology 2005, 179, 4).

Eight distinct mGluR subtypes, termed mGluR1 through mGluR8, have been identified by molecular cloning (Neuron, 1994, 13, 1031; Neuropharmacology, 1995, 34, 1; J. Med. Chem., 1995, 38, 1417). Further receptor diversity occurs via expression of alternatively spliced forms of certain mGluR subtypes (PNAS, 1992, 89, 10331; BBRC, 1994, 199, 1136; J. Neurosci., 1995, 15, 3970).

Metabotropic glutamate receptor subtypes may be subdivided into three groups, Group I, Group II, and Group III mGluRs, based on amino acid sequence homology, the second messenger systems utilized by the receptors, and by their pharmacological characteristics. Group I mGluR comprises mGluR1, mGluR5 and their alternatively spliced variants.

Attempts at elucidating the physiological roles of Group I mGluRs suggest that activation of these receptors elicits neuronal excitation. Evidence indicates that this excitation is due to direct activation of postsynaptic mGluRs, but it also has been suggested that activation of presynaptic mGluRs occurs, resulting in increased neurotransmitter release (Trends Pharmacol. Sci., 1992, 15, 92; Neurochem. Int., 1994, 24, 439; Neuropharmacology, 1995, 34, 1; Trends Pharmacol. Sci., 1994, 15, 33).

Metabotropic glutamate receptors have been implicated in a number of normal processes in the mammalian CNS. Activation of mGluRs has been shown to be required for induction of hippocampal long-term potentiation and cerebellar long-term depression (Nature, 1993, 363, 347; Nature, 1994, 368, 740; Cell, 1994, 79, 365; Cell, 1994, 79, 377). A role for mGluR activation in nociception and analgesia also has been demonstrated (Neuroreport, 1993, 4, 879; Brain Res., 1999, 871, 223).

Group I metabotropic glutamate receptors and mGluR5 in particular, have been suggested to play roles in a variety of pathophysiological processes and disorders affecting the CNS. These include stroke, head trauma, anoxic and ischemic injuries, hypoglycemia, epilepsy, neurodegenerative disorders such as Alzheimer's disease, acute and chronic pain, substance abuse and withdrawal, obesity and gastroesophageal reflux disease (GERD) (Schoepp et al., Trends Pharmacol. Sci. 1993, 14:13; Cunningham et al., Life Sci. 1994, 54:135; Hollman et al., Ann. Rev. Neurosci. 1994, 17:31; Pin et al., Neuropharmacology 1995, 34:1; Knopfel et al., J. Med. Chem. 1995, 38:1417; Spooren et al., Trends Pharmacol. Sci. 2001, 22:331; Gasparini et al. Curr. Opin. Pharmacol. 2002, 2:43; Neugebauer Pain 2002, 98:1, Slassi et al., Curr Top Med Chem. 2005; 5(9):897-911). MGluR5-selective compounds such as 2-methyl-6-(phenylethynyl)-pyridine (“MPEP”) are effective in animal models of mood disorders, including anxiety and depression (Spooren et al., J. Pharmacol. Exp. Ther. 2000, 295:1267; Tatarczynska et al., Br. J. Pharmacol. 2001, 132:1423; Klodzynska et al., Pol. J. Pharmacol, 2001, 132:1423). Much of the pathology in these conditions is thought to be due to excessive glutamate-induced excitation of CNS neurons. As Group I mGluRs appear to increase glutamate-mediated neuronal excitation via postsynaptic mechanisms and enhanced presynaptic glutamate release, their activation probably contributes to the pathology. Therefore, selective antagonists of Group I mGluR receptors could be therapeutically beneficial, especially as neuroprotective agents, analgesics or anticonvulsants.

Much of the pathology in these conditions is thought to be due to excessive glutamate-induced excitation of CNS neurons. As Group I mGluRs (mGluR1 and mGluR5) appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial, specifically as neuroprotective agents, analgesics or anticonvulsants.

German (East) Patent DD 00285356 describes 6,7- dihydro-thienopyridin derivatives and a process for their preparation.

Patent application US20050038068 describes new thienopyridone derivatives as adenosine monophosphate-activated protein kinase activators useful for the treatment of diabetes, metabolic syndrome and obesity.

Japanese Patent JP 07076586 describes furopyridines and thienopyridines as bone absorption inhibitors for the treatment of osteoporosis.

Thienopyridine derivatives are useful as hematinics, antitumor agents and immunostimulants, as described in JP 07053562 patent application.

According to E. Zeinab et al. (Arch. Pharm, 1992, 325(5), 301) thienopyridine and thienopyridones and thienopyrimidine derivatives were synthesized and their mycotoxin inhibitor activities were evaluated. Some of the compounds inhibit the production of mycotoxins and fungal growth.

International patent application WO 03/033502 describes thienopyridone derivatives which are potent inhibitors of p38 kinase and are used in the prophylaxis or treatment of p38 kinase mediated diseases, such as rheumatoid arthritis.

The compounds mentioned in the above publications are not declared or even not suggested having activity on the mGluR receptors.

### SUMMARY OF THE INVENTION

The present invention relates to new mGluR1 and mGluR5 receptor subtype preferring ligands of formula (I):

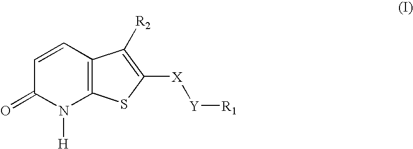

wherein
X represents a group selected from CO, SO, SO₂;
Y represents a group selected from O, OCH₂, (CH₂)ₙ, NH, NHCH₂;
n is an integer of 0 to 2;
R₁ is an optionally substituted alkyl, cycloalkyl, phenyl, biphenyl, heterocyclyl;
R₂ is an optionally substituted phenyl, heterocyclyl, or NR₃R₄ group wherein R₃ and R₄ are independently selected from the group of hydrogen, alkyl, or R₃ and R₄ together with the N atom to which they are attached can form an optionally substituted C₅₋₇ heterocyclyl group, containing one or more heteroatom(s) selected from the group of N, O, S, and/or tautomers and/or salts and/or hydrates and/or solvates thereof, to the processes for producing the same, to pharmaceutical compositions containing the same and to their use in therapy and/or prevention of pathological conditions which require the modulation of mGluR1 and mGluR5 receptors such as neurological disorders, psychiatric disorders, acute and chronic pain and neuromuscular dysfunctions of the lower urinary tract and gastrointestinal disorders.

### DETAILED DESCRIPTION OF THE INVENTION

The present invention relates to new mGluR1 and mGluR5 receptor subtype preferring ligands of formula (I):

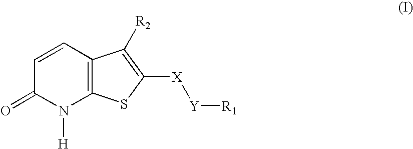

wherein
X represents a group selected from CO, SO, SO₂;
Y represents a group selected from O, OCH₂, (CH₂)ₙ, NH, NHCH₂;
n is an integer of 0 to 2;
R₁ is an optionally substituted alkyl, cycloalkyl, phenyl, biphenyl, heterocyclyl;
R₂ is an optionally substituted phenyl, heterocyclyl, or NR₃R₄ group wherein R₃ and R₄ are independently selected from the group of hydrogen, alkyl, or R₃ and R₄ together with the N atom to which they are attached can form an optionally substituted C₅₋₇ heterocyclyl group, containing one or more heteroatom(s) selected from the group of N, O, S, and/or tautomers and/or salts and/or hydrates and/or solvates thereof.

When R₁ represents alkyl, the alkyl group contains 1 to 4 carbon atom(s) with straight or branched chain, and the alkyl group may be optionally substituted with one or more substituent(s) selected from methoxy, trifluoromethyl, amino, alkylamino, dialkylamino, aminomethyl, alkylaminomethyl, dialkylaminomethyl, acylamino, cyano, fluoro, chloro, bromo.

When R₁ represents cycloalkyl, the cycloalkyl moiety contains 3 to 10 carbon atoms and may be a mono-, bi-, or tricyclic group, such as cyclohexyl or adamantyl, and the cycloalkyl group may be optionally substituted with one or more substituent(s) selected from methyl, methoxy, trifluoromethyl, amino, alkylamino, dialkylamino, aminomethyl, alkylaminomethyl, dialkylaminomethyl, acylamino, cyano, fluoro, chloro, bromo.

When R₁ and/or R₂ represents phenyl or R₁ represents biphenyl, the phenyl or biphenyl group may be optionally substituted with one or more substituent(s) selected from methyl, methoxy, trifluoromethyl, amino, alkylamino, dialkylamino, aminomethyl, alkylaminomethyl, dialkylaminomethyl, acylamino, cyano, fluoro, chloro, bromo.

When R₁ and/or R₂ represents heterocyclyl, the heterocyclic ring may be saturated or unsaturated monocyclic or bicyclic ring containing 1-4 heteroatom(s) selected from O, N or S, such as pyridyl, quinolinyl, thiazolyl, piperidinyl, morpholyl, tetrahydroquinolinyl. When the heteoatom containing ring for R₂ has no aromatic character, it must contain at least one basic nitrogen atom by which the heterocyclic group is connected with the thienopyridine moiety. The heterocyclyl group may be optionally substituted methyl, methoxy, trifluoromethyl, amino, alkylamino, dialkylamino, aminomethyl, alkylaminomethyl, dialkylaminomethyl, acylamino, cyano, fluoro, chloro, bromo.

With respect to the substituents of R₁, R₂, R₃ and R₄ the term akyl means an alkyl group containing 1 to 4 carbon atom(s) with straight or branched chain.

Depending on the circumstances compounds of formula (I) may exist in their tautomer forms (6-hydroxy-thieno[2,3-b]pyridine derivatives), too. These and their mixtures are likewise within the scope of the present invention.

Compounds of formula (I) may form salts with acids. The invention relates also to the salts of compounds of formula (I) formed with acids, especially the salts formed with pharmaceutically acceptable acids. The meaning of compound of formula (I) is either the free base or the salt even if it is not referred separately.

Both organic and inorganic acids can be used for the formation of acid addition salts. Suitable inorganic acids can be for example hydrochloric acid, sulfuric acid, nitric acid and phosphoric acid. Representatives of monovalent organic acids can be for example formic acid, acetic acid, propionic acid, and different butyric acids, valeric acids and capric acids. Representatives of bivalent organic acids can be for example oxalic acid, malonic acid, maleic acid, fumaric acid and succinic acid. Other organic acids can also be used, such as hydroxy acids for example citric acid, tartaric acid, or aromatic carboxylic acids for example benzoic acid or salicylic acid, as well as aliphatic and aromatic sulfonic acids for example methanesulfonic acid, naphthalenesulfonic acid and p-toluenesulfonic acid. Especially valuable group of the acid addition salts is in which the acid component itself is physiologically acceptable and does not have therapeutical effect in the applied dose or it does not have unfavourable influence on the effect of the active ingredient. These acid addition salts are pharmaceutically acceptable acid addition salts. The reason why acid addition salts, which do not belong to the pharmaceutically acceptable acid addition salts belong to the present invention is, that in given case they can be advantageous in the purification and isolation of the desired compounds.

Solvates and/or hydrates of compounds of formula (I) are also included within the scope of the invention.

Especially important compounds of formula (I) of the present invention are the following:

2-(4-chloro-benzenesulfonyl)-3-(4-chloro-phenyl)-7H-thieno[2,3-b]pyridin-6-one, 3-[3-(4-chloro-phenyl)-6-oxo-6,7-dihydro-thieno[2,3-b]pyridin-2-sulfonyl]-benzonitrile, 3-(4-chloro-phenyl)-6-oxo-6,7-dihydro-thieno[2,3-b]pyridine-2-carboxylic acid 4-fluoro-benzyl ester,
3-(4-chloro-phenyl)-2-(toluene-3-sulfonyl)-7H-thieno[2,3-b]pyridin-6-one, 3-(4-chloro-phenyl)-2-(3-fluoro-4-methyl-benzenesulfonyl)-7H-thieno[2,3-b]pyridin-6-one, 3-[3-(4-chloro-phenyl)-6-oxo-6,7-dihydro-thieno[2,3-b]pyridin-2-sulfonyl]-5-fluoro-benzonitrile,
3-(4-chloro-phenyl)-2-(3-fluoro-benzenesulfonyl)-7H-thieno[2,3-b]pyridin-6-one, 2-(4-chloro-benzenesulfonyl)-3-(4-methyl-piperidin- 1-yl)-7H-thieno[2,3-b]pyridin-6-one,

Pharmaceutical Formulations

The invention also relates to the pharmaceutical compositions containing the compounds of formula (I) and/or tautomers and/or physiologically acceptable salts and/or hydrates and/or solvates thereof as active ingredient and one or more physiologically acceptable carriers.

The compounds of formula (I) and/or tautomers and/or physiologically acceptable salts and/or hydrates and/or solvates thereof may be administered by any convenient method, for example by oral, parenteral (including subcutaneous, intramuscular, and intravenous), buccal, sublingual, nasal, rectal or transdermal administration and the pharmaceutical compositions adapted accordingly.

The compounds of formula (I) and/or tautomers and/or physiologically acceptable salts and/or hydrates and/or solvates thereof which are active when given orally can be formulated as liquids or solids, for example syrups, suspensions or emulsions, tablets, capsules and lozenges.

A liquid formulation of the compounds of formula (I) and/or tautomers and/or physiologically acceptable salts and/or hydrates and/or solvates thereof generally consist of a suspension or solution of the compound of formula (I) and/or tautomers and/or physiologically acceptable salts and/or hydrates and/or solvates thereof in a suitable liquid carrier(s) for example an aqueous solvent, such as water and ethanol or glycerine, or a non- aqueous solvent, such as polyethylene glycol or an oil. The formulation may also contain a suspending agent, preservative, flavouring or colouring agent.

A composition in the solid form of a tablet can be prepared using any suitable pharmaceutical carrier(s) routinely used for preparing solid formulations. Examples of solid carriers include lactose, terra alba, sucrose, talc, gelatine, agar, pectin, acacia, magnesium stearate, stearic acid etc. Optionally, tablets may be coated by standard aqueous or nonaqueous techniques.

A composition in the solid form of a capsule can be prepared using routine encapsulation procedures. For example, pellets containing the active ingredient can be prepared using standard carriers and then these are filled into a hard gelatine capsule; alternatively, a dispersion or suspension can be prepared using any suitable pharmaceutical carrier(s), for example aqueous gums, celluloses, silicates or oils and the dispersion or suspension then is filled into a soft gelatine capsule.

Typical parenteral compositions consist of a solution or suspension of the compound of formula (I) and/or tautomers and/or physiologically acceptable salts and/or hydrates and/or solvates thereof in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions of the present invention for nasal administration containing a compound of formula (I) and/or tautomers and/or physiologically acceptable salts and/or hydrates and/or solvates thereof may conveniently be formulated as aerosols, drops, gels and powders. Aerosol formulations of the present invention typically comprise a solution or fine suspension of the compound of formula (I) and/or tautomers and/or physiologically acceptable salts and/or hydrates and/or solvates in a physiologically acceptable aqueous or non-aqueous solvent and are usually presented in a single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal once the contents of the container have been exhausted. If the dosage form comprises an aerosol dispenser, it will contain a propellant which can be a compressed gas, such as compressed air or an organic propellant, such as a fluorochlorohydrocarbon. The aerosol dosages form can also take the form of a pump-atomiser.

Compositions of the present invention containing a compound of formula (I) and/or tautomers and/or physiologically acceptable salts and/or hydrates and/or solvates are suitable for buccal or sublingual administration including tablets, lozenges and pastilles, wherein the active ingredient is formulated with a carrier, such as sugar and acacia, tragacanth, or gelatine, glycerin etc.

Compositions of the present invention containing a compound of formula (I) and/or tautomers and/or physiologically acceptable salts and/or hydrates and/or solvates thereof for rectal administration are conveniently in the form of suppositories containing a conventional suppository base, such as cocoa butter and other materials commonly used in the art. The suppositories may be conveniently formed by first admixing the composition with the softened or melted carrier(s) followed by chilling and shaping in moulds.

Compositions of the present invention containing a compound of formula (I) and/or tautomers and/or physiologically acceptable salts and/or hydrates and/or solvates thereof for transdermal administration include ointments, gels and patches.

The compositions of the present invention containing a compound of formula (I) and/or tautomers and/or physiologically acceptable salts and/or hydrates and/or solvates thereof is preferably in the unit dose form, such as tablet, capsule or ampoule.

Each dosage unit of the present invention for oral administration contains preferably from 0.1 to 500 mg of a compound of formula (I) and/or tautomers and/or physiologically acceptable salts and/or hydrates and/or solvates thereof calculated as a free base.

Each dosage unit of the present invention for parenteral administration contains preferably from 0.1 to 500 mg of a compound of formula (I) and/or tautomers and/or physiologically acceptable salts and/or hydrates and/or solvates thereof calculated as a free base.

The compounds of formula (I) and/or tautomers and/or physiologically acceptable salts and/or hydrates and/or solvates thereof can normally be administered in a daily dosage regimen. In the treatment of mGluR1 and mGluR5 mediated disorders, such as schizophrenia, anxiety, depression, panic, bipolar disorders, and circadian disorders or chronic and acute pain disorders the dosage levels from about 0.01 mg/kg to about 140 mg/kg of body weight per day are useful or alternatively about 0.5 mg to about 7 g per patient per day.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms will generally contain between from about 1 mg to about 1000 mg of the active ingredient, typically 25 mg, 50 mg, 100 mg, 200 mg, 250-300 mg, 400 mg, 500 mg, 600 mg, 800 mg or 1000 mg.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

Medical Use

The compounds of formula (I) and/or tautomers and/or physiologically acceptable salts and/or hydrates and/or solvates of the present invention have been found to exhibit biological activity at mGluR1 and mGluR5 receptors and are expected to be useful in the treatment of mGluR1 and mGluR5 mediated disorders.

It has been found that the compounds according to the present invention or salts thereof, exhibit a high degree of potency and selectivity for individual metabotropic glutamate receptor (mGluR) subtypes. In particular there are compounds according to the present invention that are potent and selective for mGluR1 and mGluR5 receptors. Accordingly, the compounds of the present invention are expected to be useful in the prevention and/or treatment of conditions associated with excitatory activation of mGluR1 and mGluR5 receptor and for inhibiting neuronal damage caused by excitatory activation of mGluR1 and mGluR5 receptor. The compounds may be used to produce an inhibitory effect of mGluR1 and mGluR5, in mammals, including human.

Thus, it is expected that the compounds of the invention are well suited for the prevention and/or treatment of mGluR1 and mGluR5 receptor-mediated disorders such as acute and chronic neurological and psychiatric disorders, chronic and acute pain disorders, neuromuscular dysfunctions of the lower urinary tract and gastrointestinal disorders.

The dose required for the therapeutic or preventive treatment of a particular disorder will necessarily be varied depending on the host treated and the route of administration.

The invention relates to compounds of formula (I) as defined hereinbefore, for use in therapy.

The invention relates to compounds of formula (I) as defined hereinbefore, for use in prevention and/or treatment of mGluR1 and mGluR5 receptor-mediated disorders.

The invention relates to compounds of formula (I) as defined hereinbefore, for use in prevention and/or treatment of neurological disorders.

The invention relates to compounds of formula (I) as defined hereinbefore, for use in prevention and/or treatment of psychiatric disorders.

The invention relates to compounds of formula (I) as defined hereinbefore, for use in prevention and/or treatment of chronic and acute pain disorders.

The invention relates to compounds of formula (I) as defined hereinbefore, for use in prevention and/or treatment of neuromuscular dysfunctions of the lower urinary tract and gastrointestinal disorders.

The invention relates to compounds of formula (I) as defined hereinbefore, for use in prevention and/or treatment of pain related to migraine, inflammatory pain, neuropathic pain disorders such as diabetic neuropathies, arthritis and rheumatoid diseases, low back pain, post-operative pain and pain associated with various conditions including angina, in renal or biliary colic, menstruation, migraine and gout.

The invention relates to compounds of formula (I) as defined hereinbefore, for use in prevention and/or treatment of Alzheimer's disease senile dementia, AIDS-induced dementia Parkinson's disease, amyotrophic lateral sclerosis, Huntington's Chorea, migraine, epilepsy, schizophrenia, depression, anxiety, acute anxiety, obesity, obsessive compulsive disorder, ophthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, Fragile X, autism, mental retardation, schizophrenia and Down's Syndrome.

The invention relates to compounds of formula (I) as defined hereinbefore, for use in prevention and/or treatment of stroke, head trauma, anoxic and ischemic injuries, hypoglycemia, cardiovascular diseases and epilepsy.

The compounds are also well suited for the treatment of neuromuscular dysfunction of the lower urinary tract, such as urinary urgency, overactive bladder, greater urinary frequency, reduced urinary compliance, cystitis, incontinence, enuresis and dysuria.

The compounds are also well suited for the treatment of gastrointestinal disorders, such as transient lower esophageal sphincter relaxation (TLESR), gastrointestinal reflux disease and irritable bowel syndrome.

The present invention relates also to the use of a compound of formula (I) as defined hereinbefore, in the manufacture of a medicament for the prevention and/or treatment of mGluR1 and mGluR5 receptor-mediated disorders and any disorder listed above.

The invention also provides a method of treatment and/or prevention of mGluR1 and mGluR5 receptor mediated disorders and any disorder listed above, in a patient suffering from, or at risk of, said condition, which comprises administering to the patient an effective amount of a compound of formula (I), as hereinbefore defined.

In the context of the present specification, the term “therapy” includes treatment as well as prevention, unless there are specific indications to the contrary. The terms “therapeutic” and “therapeutically” should be construed accordingly.

In this specification, unless stated otherwise, the term “antagonist” means a compound that by any means, partly or completely blocks the transduction pathway leading to the production of a response by the ligand.

The term “disorder”, unless stated otherwise, means any condition and disease associated with metabotropic glutamate receptor activity.

Methods of preparation

Abbreviation

The abbreviation used herein has the following tabulated meaning. Abbreviations not tabulated below have their meanings as commonly used unless specifically stated otherwise.

DMF N,N-dimethylformamide

Compounds of the present invention can be prepared according to the following methods. Unless stated otherwise, the meaning of substituents is as defined above for formula I or apparent to one skilled in the art.

According to the present invention a process for the preparation of a compound of formula (I)

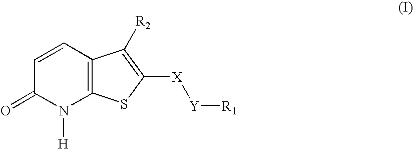

wherein

X represents a group selected from CO, SO, SO₂;

Y represents a group selected from O, OCH₂, (CH₂)ₙ, NH, NHCH₂;

n is an integer of 0 to 2;

R₁ is an optionally substituted alkyl, cycloalkyl, phenyl, biphenyl, heterocyclyl;

R₂ is an optionally substituted phenyl, heterocyclyl, and/or tautomers and/or salts and/or hydrates and/or solvates thereof, reacting a thienopyridine derivative of formula (II)

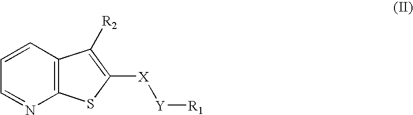

wherein the meaning of X, Y, R₁ and R₂ are as defined above for a compound of formula (I), with m-chloroperoxybenzoic acid or peroxyacetic acid in a solvent, to obtain compound of formula (III)

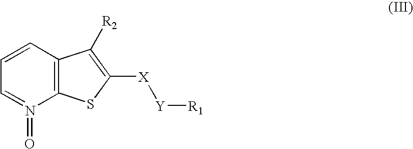

wherein the meaning of X, Y, R₁ and R₂ are as defined above for a compound of formula (I), thereafter reacting the compound of formula (III) with trifluoroacetic anhydride or acetic anhydride in dimethylformamide to give a compound of formula (I) and optionally thereafter forming tautomers and/or salts and/or hydrates and/or solvates of compounds of formula (I) or reacting a thienopyridine derivative of formula (IV)

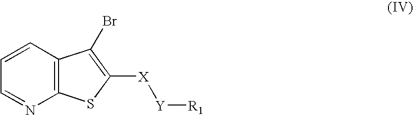

wherein the meaning of X, Y and R₁ are as defined above for the formula (I) with m-chloroperoxybenzoic acid or peroxyacetic acid in a solvent to obtain a compound of formula (V)

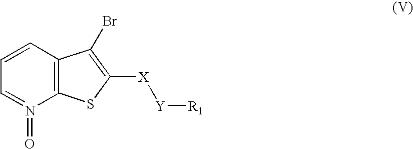

wherein the meaning of X, Y and R₁ are as defined above for the formula (I), thereafter coupling a compound of formula (V) with a compound of formula (VI)

R₂—B(OH)₂ (VI)

wherein R₂ is an optionally substituted phenyl or heterocyclyl, to obtain a compound of formula (III)

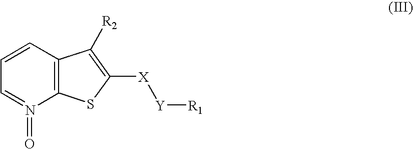

wherein the meaning of X, Y, R₁ and R₂ are as defined above for the formula (I) and coupling a compound of formula (III) with trifluoroacetic anhydride or acetic anhydride in dimethylformamide to obtain a compound of formula (I) and optionally thereafter forming tauromers and/or salts and/or hydrates and/or solvates of compounds of formula (I).

Another process for the preparation of a compound of formula (I)

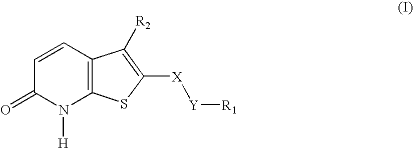

wherein

X represents a group selected from CO, SO, SO₂;

Y represents a group selected from O, OCH₂, (CH₂)ₙ, NH, NHCH₂;

n is an integer of O to 2;

R₁ is an optionally substituted alkyl, cycloalkyl, phenyl, biphenyl, heterocyclyl;

R₂ is an optionally substituted NR₃R₄ group wherein R₃ and R₄ are independently selected from the group of hydrogen, alkyl, or R₃ and R₄ together with the N atom to which they are attached can form an optionally substituted C₅₋₇ heterocyclyl group, containing one or more heteroatom(s) selected from the group of N, O, S and/or tautomers and/or salts and/or hydrates and/or solvates thereof coupling the compound of formula (V)

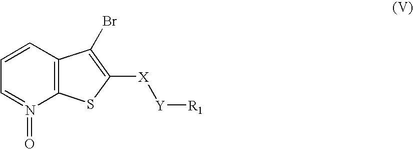

wherein the meaning of X, Y and R₁ are as defined above for the formula (I) with a compound of formula (VII)

HNR₃R₄ (VII)

wherein R₃ and R₄ are independently selected from the group of hydrogen, alkyl, or R₃ and R₄ together with the N atom to which they are attached can from an optionally substituted C₅₋₇ heterocyclyl group, containing one or more heteroatom(s) selected from the group of N, O, S, in dimethylformamide to obtain a compound of formula (III)

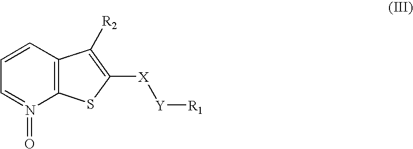

wherein the meaning of X, Y, R₁ and R₂ are as defined above for the formula (I) and reacting a compound of formula (II) with trifluoroacetic anhydride or acetic anhydride in dimethylformamide to obtain a compound of formula (I) and optionally thereafter forming tauromers and/or salts and/or hydrates and/or solvates of compounds of formula (I).

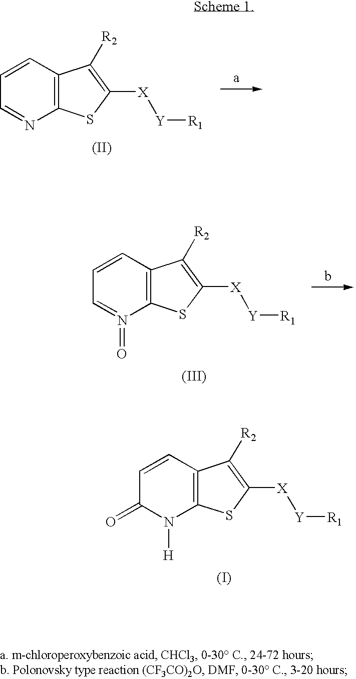

Compound of formula (II) was prepared according to the method described in Hungarian Patent Application P0501168 or P0501171.

N-oxide derivatives of formula (III) were prepared from compounds of formula (II) by oxidation with e.g. m-chloroperoxybenzoic acid or peroxyacetic acid in an appropriate solvent (chloroform, dichloromethane, acetic acid, etc.) at ambient temperature by the method of W. Boisvert (J. Heterocyclic Chem., 1987, 24, 1467).

Compounds of formula (III) were treated with trifluoroacetic anhydride or with acetic anhydride in dimethylformamide by a Polonovsky type reaction using the method of R. Hartling (J. Heterocyclic Chem., 1976, 13, 1197). The reaction was carried out at ambient temperature and resulted in the compounds of formula (I).

The obtained compounds of formula (I) can be purified by crystallization or by column chromatography.

The process according Scheme 1 is useful for the preparation of compounds of formula (I) wherein R₂ is an optionally substituted phenyl or heterocyclyl.

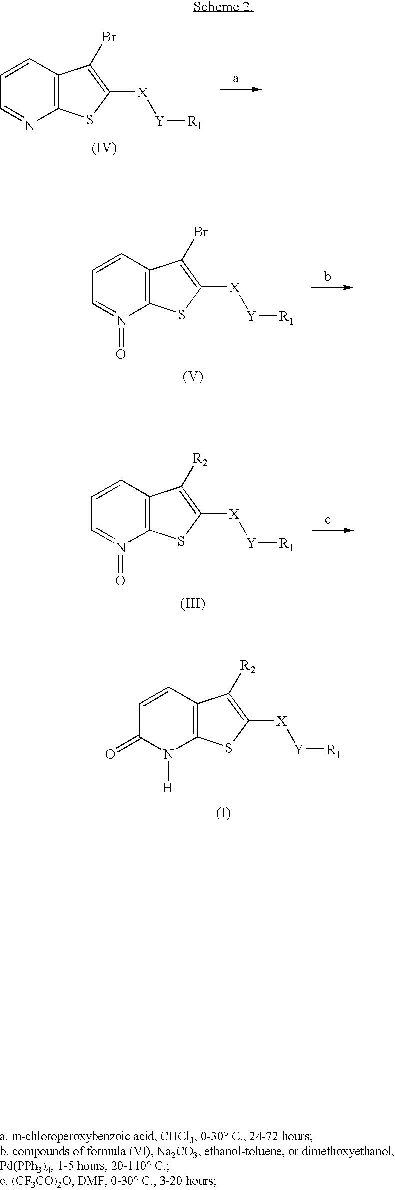

Compound of formula (IV) was prepared according to the method described in Hungarian Patent Application P0501171.

N-oxide derivatives of formula (V) were prepared from compounds of formula (IV) by oxidation with e.g. m-chloroperoxybenzoic acid or peroxyacetic acid in an appropriate solvent (chloroform, dichloromethane, acetic acid, etc.) at ambient temperature by the method of W. Boisvert (J. Heterocyclic Chem., 1987, 24, 1467).

Compounds of formula (III) were synthesized from compounds of formula (V) and (VI) by the well known methods of the Suzuki coupling reactions (A. Suzuki & H. C. Brown: Organic Syntheses via Boranes Vol. 1-3).

Compounds of formula (III) were treated with trifluoroacetic anhydride or with acetic anhydride in dimethylformamide by a Polonovsky type reaction using the method of R. Hartling (J. Heterocyclic Chem., 1976, 13, 1197). The reaction was carried out at ambient temperature and resulted in the compounds of formula (I).

The obtained compounds of formula (I) can be purified by crystallization or by column chromatography.

The process according Scheme 2 is useful for the preparation of compounds of formula (I) wherein R₂ is an optionally substituted phenyl or heterocyclyl.

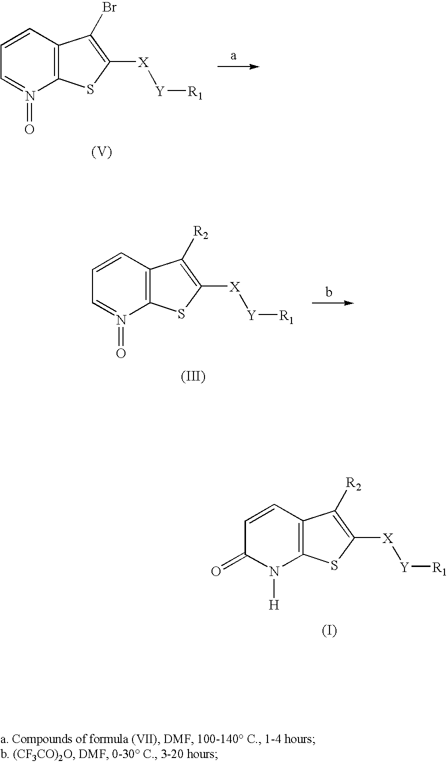

Compounds of formula (III) were prepared from compounds of formula (V) by the modified methods of the D. Prim and G. Kirsch (Tetrahedron, 1999, 55, 6511-6526).

Compounds of formula (III) were treated with trifluoroacetic anhydride or with acetic anhydride in dimethylformamide by a Polonovsky type reaction using the method of R. Hartling (J. Heterocyclic Chem., 1976, 13, 1197). The reaction was carried out at ambient temperature and resulted in the compounds of formula (I).

The obtained compounds of formula (I) can be purified by crystallization or by column chromatography.

The process according Scheme 3 is useful for the preparation of compounds of formula (I) wherein R₂ is an optionally substituted NR₃R₄ group wherein R₃ and R₄ are independently selected from the group of hydrogen, alkyl, or R₃ and R₄ together with the N atom to which they are attached can form an optionally substituted C₅₋₇ heterocyclyl group, containing one or more heteroatom(s) selected from the group of N, O, S.

Biological Test Methods

MGluR1 Receptor Binding Test

MGluR1 receptor binding tests were performed according to modified method of Lavreysen et al. (Mol.Pharm., 2003, 63, 1082). Based on the high homology between the human and rat mGluR1 receptors, rat cerebellar membrane preaparation was used to determine the binding characteristics of reference compounds and novel compounds to the rat mGluR1. As radioligand [3H]R214127 (3 nM) was used and the nonspecific binding was determined in the presence of 1 μM of R214127.

IC-50 values were determined from displacement curves by nonlinear regression analysis and were converted by equation method of Cheng and Prusoff (Biochem. Pharmacol., 1973, 22, 3099) to Ki values.

MGluR5 Receptor Binding Tests

MGluR5 receptor binding was determined according to Gasparini et.al. (Bioorg. Med. Chem. Lett. 2000, 12:407-409) with modifications. Rat cerebro-cortical membrane preparation was used to determine the binding characteristics of reference compounds and novel compounds to the rat mGluR5. The A18 cell line expressing hmGluR5a (purchased from Euroscreen) was used to determine binding characteristics of the chemical compounds to the human mGluR5a receptor. As radioligand [3H]-M-MPEP (2 nM) was used. The nonspecific binding was determined in the presence of 10 μM M-MPEP.

Assessment of Functional Activity

Cell Cultures for Native Rat mGluR5 and mGluR1 Receptors

Functional potency at native rat mGluR5 and mGluR1 receptors was estimated using primary neocortical cell cultures derived from 17 day old Charles River rat embryos and primary cerebellar cell cultures derived from 4-day old Wistar rats, respectively (for the details on the preparation of neural cell cultures see Johnson, M. I.; Bunge, R. P. (1992): Primary cell cultures of peripheral and central neurons and glia. In: Protocols for Neural Cell Culture, eds: Fedoroff, S.; Richardson A., The Humana Press Inc., 51-77). After isolation the cells were plated onto standard 96-well microplates and the cultures were maintained in an atmosphere of 95% air-5% CO₂ at 37° C. The neocortical and cerebellar cultures were used for the calcium measurements after 5-7 and 3-4 days in vitro, respectively.

Cell Cultures for Recombinant Human mGluR5a Receptors

Chinese hamster ovary (CHO) cells stably expressing recombinant human mGluR5a (CHO-mGluR5a, purchased from Euroscreen) receptors were cultured in F12 medium containing 10% FCS, 1% antibiotic antimycotic solution, 400 μg/ml G418, 250 μg/ml zeocin, 5 μg/ml puromycin. Cells were kept at 37° C. in a humidified incubator in an atmosphere of 5% CO₂/95% air and were passaged three times a week. Cells were plated at 2.5-3.5×104 cell/well on standard 96-well microplates, receptor expression was induced by adding 600 ng/ml doxycycline on the next day. The calcium measurements were carried out 16-24 hours after the addition of the inducing agent.

Fluorimetric Measurement of Cytosolic Calcium Concentration

Measurements of cytosolic calcium concentration ([Ca²⁺]ᵢ) were carried out on primary neocortical and cerebellar cultures, and on CHO-mGluR5a cells stably expressing human mGluR5a receptors. Cells were grown in standard 96-well microplates and before the measurement were loaded with a fluorescent Ca²⁺-sensitive dye, fluo-4/AM (2 μM): the neural cultures were loaded in their growth medium, CHO-mGluR5a cells were loaded in assay buffer (145 mM NaCl, 5 mM KCl, 2 mM MgCl₂, 2 mM CaCl₂, 10 mM HEPES, 20 mM D-glucose, 2 mM probenecid, pH=7.4) supplemented with 2 mM Na-pyruvate and 30 μg/ml glutamate-pyruvate transaminase (in case of CHO-mGluR5a cells these supplements were also present during the course of the [Ca²⁺]ᵢ measurements). Loading was done by incubating the cells with 100 μl/well dye solution at 37° C. in a humidified incubator in an atmosphere of 5% CO2/95% air for 40-120 min. To stop dye loading cells were washed twice with assay buffer. After washing, various concentrations of the test compounds (diluted in assay buffer from a DMSO or a dimethylformamide (DMF) stock solution, final DMSO/DMF concentration was <0.1%) or buffer were added to each well depending on the experimental setup. In the case of neocortical cultures the assay buffer also contained TTX (0.5 μM, to suppress spontaneous oscillations of [Ca2+]i, in the case of cerebellar cultures probenecid was substituted with sulfinpyrazone (0.25 mM).

After incubation at 37° C. for 10-20 min. baseline and agonist-evoked changes of [Ca2+]i were measured column by column with a plate reader fluorimeter (FlexStation II, Molecular Devices). Excitation and detection of emission was carried out from the bottom of the plate. The whole measurement process was performed at 37° C. and was controlled by custom software. Inhibitory potency of the test compounds was assessed by measuring the reduction in the agonist-evoked [Ca²⁺]ᵢ-elevation in the presence of different concentrations of the compounds. DHPG was used as agonist for all three cultures, the concentration was 20 and 100 μM for neocortical and cerebellar cultures, respectively. In the case of CHO-mGluR5a cells DHPG was applied at an EC80 concentration, the EC80-values were derived from daily determined dose-response curves. Fluorescence data were expressed as ΔF/F (fluorescence change normalized to baseline).

All treatments on a single plate were measured in multiple wells. Data from all wells with the same treatment were averaged and the average values were used for analysis. Inhibitory potency of a compound at a single concentration point was expressed as percent inhibition of the control agonist response. Sigmoidal concentration-inhibition curves were fitted to the data (derived from at least three independent experiments) and IC50-values were determined as the concentration that produces half of the maximal inhibition caused by the compound. Raw fluorescence data were analyzed using Soft Max Pro (Molecular Devices), curve fitting was done with GraphPad Prism.

Results

Compounds of formula (I) of the present invention showed affinity for both rat and human mGluR1 and mGluR5 receptors and proved to be functional antagonists that are they inhibited functional responses elicited by stimulation of mGluR5 receptors.

TABLE

| | | | mGlu5 | mGlu1 | |
|---|---|---|---|---|---|
| Comp. | | | Kᵢ | Kᵢ | |
| No. | Structure | (M + H)⁺ | (nM) | (nM) | ¹H NMR data |
| 1 | 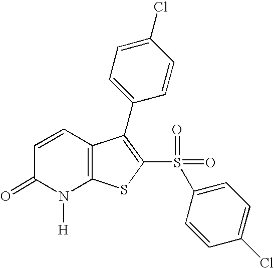 | 437.2 | * | ** | (500 MHz, DMSO-d₆, 25° C.): 12.53 (vbrs, 1H); 7.60-7.51 (m, 4H); 7.50-7.45 (m, 2H); 7.45-7.33 (brm, 1H); 7.25- 7.19 (m, 2H); 6.70-6.37 (brm, 1H). |
| 2 | 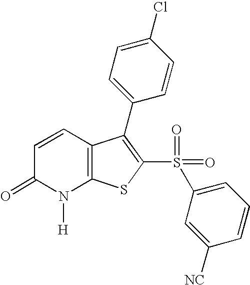 | 427.3 | * | * | (500 MHz, DMSO-d₆, 50° C.): 12.39 (vbrs, 1H); 8.07 (dm, J = 7.8 Hz, 1H); 7.80 (dm, J = 8.0 Hz, 1H); 7.71-7.63 (m, 2H); 7.53-7.48 (m, 2H); 7.39 (d, J = 9.3 Hz, 1H); 7.21- 7.13 (m, 2H); 6.55 (brd, J = 9.3 Hz, 1H). |
| 3 | 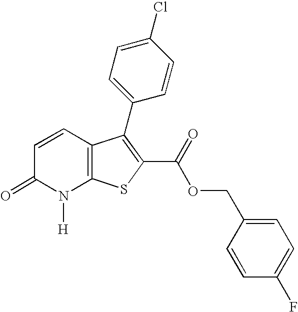 | 414.2 | * | * | (500 MHz, DMSO-d₆, 50° C.): 12.33 (vbrs, 1H); 7.50-7.41 (m, 3H); 7.40-7.35 (m, 2H); 7.26-7.20 (m, 2H); 7.17-7.10 (m, 2H); 6.52 (brd, J = 8 Hz, 1H). |
| 4 | 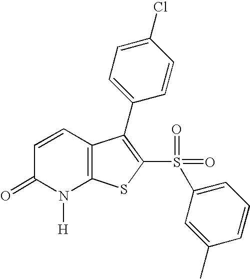 | 416.2 | ** | | (500 MHz, DMSO-d₆, 25° C.): 12.47 (vbrs, 1H); 7.58-7.51 (m, 2H); 7.44 (dm, J = 7.9 Hz, 1H); 7.40 (brs, 1H); 7.37 (t, J = 7.9 Hz, 1H); 7.32 (dm, J = 7.9 Hz, 1H); 7.21-7.16 (m, 2H); 7.11-7.07 (m, 1H); 6.55 (brs, 1H); 2.25 (s, 3H). |
| 5 | 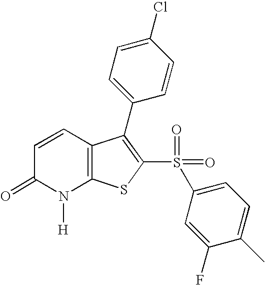 | 434 | | | |
| 6 | 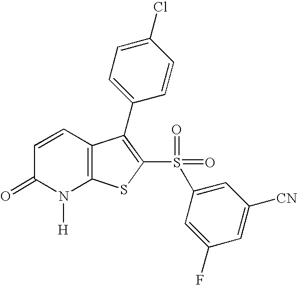 | 445.2 | | | |
| 7 | 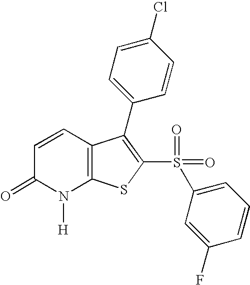 | 420.2 | | | |
| 8 | 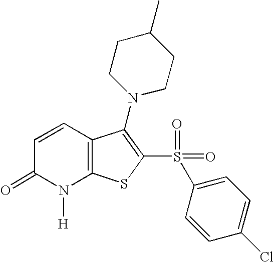 | 423.2 | | | |

* Kᵢ < 500 nM
** Kᵢ > 500 nM

The invention is further illustrated by the following non-limiting examples.

### EXAMPLES

### Example 1

2-(4-Chloro-Benzenesulfonyl)-3-(4-Chloro-Phenyl)-Thieno[2,3-b]Pyridine-N-oxide

To the solution of 2-(4-chloro-benzenesulfonyl)-3-(4-chloro-phenyl)-thieno[2,3-b]pyridine (2.35 g, 5.6 mmol) in chloroform (50 ml) m-chloroperoxybenzoic acid (2.52 g, 11.2 mmol, 77%) was added and the reaction mixture was stirred at room temperature for two days. Solution of NaHCO₃ (10%, 30 ml) was then added and after separation of the phases the organic phase was washed with water (10 ml), dried over Na₂SO₄, filtered, and contcentrated in vacuo. The crude product was purified by a treatment with ether-chloroform (1:1) to give 1.9 g (78%) of the title compound. MS: m/e=437.2 (M+H)⁺.

### Example 2

2-(4-Chloro-Benzenesulfonyl)-3-(4-Chloro-Phenyl)-7H-Thieno[2,3-b]Pyridin-6-One

(Compound 1)

2-(4-Chloro-benzenesulfonyl)-3-(4-chloro-phenyl)-thieno[2,3-b]pyridine-N-oxide 0.57 g, 1.3 mmol) was treated in DMF (6 ml) suspension with trifluoroacetic anhydrate (5.5 ml, 39.5 mmol) at room temperature for 7 hours. By the end of the reaction it became a solution, which was concentrated by half of the whole volume in vacuo. Water (0.3 ml) was added, the crystalline product was filtered off and washed with water-DMF mixture (1:1). The reaction resulted in 0.35 g (61%) of the titled compound. MS: m/e=437.2 (M+H)⁺. The preparations of compounds 2 and 3 were as described above.

### Example 3

3-(3-Bromo-Thieno[2,3-b]Pyridine-2-Sulfonyl)-5-Fluoro-Benzonitrile-N-Oxide

To the solution of 3-(3-bromo-thieno[2,3-b]pyridine-2-sulfonyl)-5-fluoro-benzonitrile (3.51 g, 8.8 mmol) in chloroform (130 ml) m-chloroperoxybenzoic acid (6.08 g, 35 mmol, 77%) was added and the reaction mixture was stirred at room temperature overnight. The solution was washed three times with NaHCO₃ (10%, 50 ml) then twice with water (25 ml), dried over Na₂SO₄, filtered and concentrated in vacuo. The crude product was purified by a treatment with ether to give 3.06 g (84%) of the title compound. MS: m/e=414.1 (M+H)⁺.

### Example 4

3-[3-(4-Chloro-Phenyl)-Thieno[2,3-b]Pyridine-2-Sulfonyl]-5-Fluoro-Benzonitrile-N-Oxide

3-(3-Bromo-thieno[2,3-b]pyridine-2-sulfonyl)-5-fluoro-benzonitrile-N-oxide (Example 3) (2.1 g, 5 mmol) was dissolved in toluene (40 ml) and ethanol (45 ml) under argon atmosphere. To the solution Pd(PPh₃)₄ (0.28 g, 0.25 mmol), 4-chlorophenylboronic acid (0.94 g, 6 mmol) and 2M solution of Na₂CO₃ (23 ml) was added, then reaction mixture was refluxed for one hour. Water (50 ml) was added and the obtained suspension was extracted three times with ethyl acetate (50 ml). The organic phase was washed with water (50 ml), dried over Na₂SO₄, filtered and contcentrated in vacuo. The crude product was purified by column chromatography (Kieselgel 60, eluent: ethyl acetate : n-hexane=5:1) to yield 1.26 g (56 %) of the title compound. MS: m/e=445.2 (M+H)⁺.

### Example 5

3-[3-(4-Chloro-Phenyl)-6-Oxo-6,7-Dihydro-Thieno[2,3-b]Pyridine-2-Sulfonyl]-5-Fluoro-Benzonitrile

(Compound 6)

3-[3-(4-Chloro-phenyl)-thieno[2,3 -b]pyridine-2-sulfonyl]-5-fluoro-benzonitrile-N-oxide (Example 4) (0.94 g, 2 mmol) was treated in DMF (8 ml) suspension with trifluoroacetic anhydride (6 ml, 43 mmol) at room temperature for 2 hours. By the end of the reaction it became a solution, which was concentrated by half of the whole volume in vacuo. Water (6 ml) was added, the crystalline product was filtered off and washed with water-DMF mixture (1:1). The reaction resulted in 0.7 g (74%) of the titled compound. MS: m/e=445.2 (M+H)⁺.

Compounds 4, 5 and 7 were prepared according to the above described method.

### Example 6

2-(4-Chloro-Benzenesulfonyl)-3-(4-Methyl-Piperidin-1-yl)-Thieno[2,3-b]Pyridine-N-Oxide

3-(3-Bromo-thieno[2,3-b]pyridine-2-sulfonyl)-5-fluoro-benzonitrile-N-oxide (Example 3) (4.1 g, 10 mmol) and 4-methyl-piperidine (6 ml, 50 mmol) in DMF (30 ml) was stirred at 80° C. for 2 hours. The solution was concentrated in vacuo and the residue was suspended in water (100 ml). The precipitate was filtered and washed with water (3×20 ml) to yield 3.76 g (92 %) of the title compound. MS: m/e=423.1 (M+H)⁺.

### Example 7

3-[3-(4-Chloro-Phenyl)-6-Oxo-6,7-Dihydro-Thieno[2,3-b]Pyridine-2-Sulfonyl]-5-Fluoro-Benzonitrile

(Compound 8)

The title compound was prepared from 2-(4-Chloro-benzenesulfonyl)-3-(4-methyl-piperidin-1-yl)-thieno[2,3-b]pyridine-N-oxide (Example 6) according to the method described in Example 5. MS: m/e=423.2 (M+H)⁺.

### Example 8

Preparation of Pharmaceutical Compositions:

### a) Tablets:

0.01-50 % of active ingredient of formula (I), 15-50 % of lactose, 15-50 % of potato starch, 5-15% of polyvinyl pyrrolidone, 1-5% of talc, 0.01-3% of magnesium stearate, 1-3% of colloid silicon dioxide and 2-7% of ultraamylopectin were mixed, then granulated by wet granulation and pressed to tablets.

b) Dragées, Filmcoated Tablets:

The tablets made according to the method described above were coated by a layer consisting of entero- or gastrosolvent film, or of sugar and talc. The dragées were polished by a mixture of beeswax and carnuba wax.

### c) Capsules:

0.01-50% of active ingredient of formula (I), 1-5% of sodium lauryl sulfate, 15-50% of starch, 15-50% of lactose, 1-3% of colloid silicon dioxide and 0.01-3% of magnesium stearate were thoroughly mixed, the mixture was passed through a sieve and filled in hard gelatin capsules.

### d) Suspensions:

Ingredients: 0.01-15% of active ingredient of formula (I), 0.1-2% of sodium hydroxide, 0.1-3% of citric acid, 0.05-0.2% of nipagin (sodium methyl 4-hydroxybenzoate), 0.005-0.02% of nipasol, 0.01-0.5% of carbopol (polyacrilic acid), 0.1-5% of 96% ethanol, 0.1-1% of flavoring agent, 20-70% of sorbitol (70% aqueous solution) and 30-50% of distilled water.

To solution of nipagin and citric acid in 20 ml of distilled water, carbopol was added in small portions under vigorous stirring, and the solution was left to stand for 10-12 h. Then the sodium hydroxide in 1 ml of distilled water, the aqueous solution of sorbitol and finally the ethanolic raspberry flavor were added with stirring. To this carrier the active ingredient was added in small portions and suspended with an immersing homogenizator. Finally the suspension was filled up to the desired final volume with distilled water and the suspension syrup was passed through a colloid milling equipment.

### e) Suppositories:

For each suppository 0.01-15% of active ingredient of formula (I) and 1-20% of lactose were thoroughly mixed, then 50-95% of adeps pro suppository (for example Witepsol 4) was melted, cooled to 35° C. and the mixture of active ingredient and lactose was mixed in it with homogenizator. The obtained mixture was mould in cooled forms.

f) Lyophilized powder ampoule compositions:

A 5% solution of mannitol or lactose was made with bidistilled water for injection use, and the solution was filtered so as to have sterile solution. A 0.01-5% solution of the active ingredient of formula (I) was also made with bidistilled water for injection use, and this solution was filtered so as to have sterile solution. These two solutions were mixed under aseptic conditions, filled in 1 ml portions into ampoules, the content of the ampoules was lyophilized, and the ampoules were sealed under nitrogen. The contents of the ampoules were dissolved in sterile water or 0.9% (physiological) sterile aqueous sodium chloride solution before administration.

1-12. (canceled)

13. A compound of formula (I): 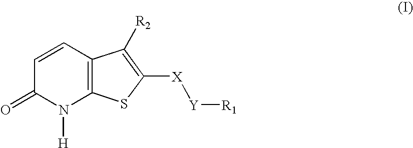
wherein:
X is selected from CO, SO, and SO₂;
Y is selected from O, OCH₂, (CH2)ₙ, NH, and NHCH₂;
n is an integer ranging from 0 to 2;
R₁ is an optionally substituted alkyl, cycloalkyl, phenyl, biphenyl, or heterocyclyl;
R₂ is an optionally substituted phenyl, heterocyclyl or NR₃R₄ group, wherein R₃ and R₄ are independently selected from hydrogen, and alkyl, or R₃ and R₄ together with the N atom to which they are attached can form an optionally substituted C₅₋₇ heterocyclyl group containing one or more heteroatom(s) selected from the group of N, O, and S; or tautomers, salts, hydrates, or solvates thereof.

14. A compound selected from:
2-(4-chloro-benzenesulfonyl)-3-(4-chloro-phenyl)-7H-thieno[2,3-b] yridine-6-one, 3-[3-(4-chloro-phenyl)-6-oxo-6,7-dihydro-thieno[2,3-b]pyridin-2-sulfonyl]-benzonitrile, 3-(4-chloro-phenyl)-6-oxo-6,7-dihydro-thieno[2,3-b]pyridine-2-carboxylic acid 4-fluoro-benzyl ester,
3-(4-chloro-phenyl)-2-(toluene-3-sulfonyl)-7H-thieno[2,3 -b]pyridine-6-one, 3-(4-chloro-phenyl)-2-(3-fluoro-4-methyl-benzenesulfonyl)-7H-thieno[2,3-b]pyridine-6-one, 3-[3-(4-chloro-phenyl)-6-oxo-6,7-dihydro-thieno [2,3-b]pyridin-2-sulfonyl]-5-fluoro-benzonitrile,
3-(4-chloro-phenyl)-2-(3-fluoro-benzenesulfonyl)-7H-thieno [2,3-b]pyridine-6-one, and 2-(4-chloro-benzenesulfonyl)-3-(4-methyl-piperidin-1-yl)-7H-thieno[2,3 -b]pyridine-6-one.

15. A process for the preparation of a compound of formula (I): 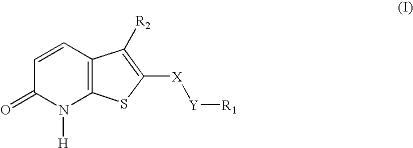
wherein:
X is selected from CO, SO, and SO₂;
Y is selected from O, OCH₂, (CH₂)ₙ, NH, and NHCH₂;
n is an integer ranging from 0 to 2;
R₁ is an optionally substituted alkyl, cycloalkyl, phenyl, biphenyl, or heterocyclyl;
R₂ is an optionally substituted phenyl or heterocyclyl; or tautomers, salts, hydrates, or solvates thereof, comprising:
a.) reacting a thienopyridine derivative of formula (II): 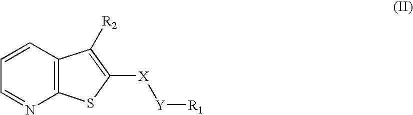
wherein the meaning of X, Y, R₁ and R₂ are as defined above for the compound of formula (I), with m-chloroperoxybenzoic acid or peroxyacetic acid in a solvent, to obtain compound of formula (III): 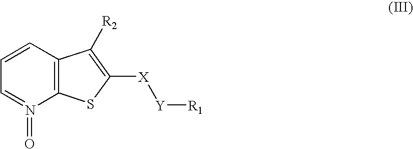
wherein the meaning of X, Y, R₁ and R₂ are as defined above for a compound of formula (I); thereafter reacting the compound of formula (III) with trifluoroacetic anhydride or acetic anhydride in dimethylformamide to give a compound of formula (I); and, optionally thereafter forming one or more tautomers, salts, hydrates or solvates of compounds of formula (I); or,
b.) reacting a thienopyridine derivative of formula (IV): 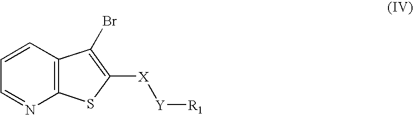
wherein the meaning of X, Y and R₁ are as defined above for the formula (I) with m-chloroperoxybenzoic acid or peroxyacetic acid in a solvent to obtain a compound of formula (V): 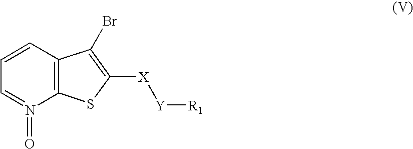
wherein the meaning X, Y, and R₁ are as defined above for the formula (I); thereafter coupling the compound of formula (V) with a compound of formula (VI):
R2—B(OH)₂ (VI)
wherein R2 is an optionally substituted phenyl or heterocyclyl, to obtain a compound of formula (III): 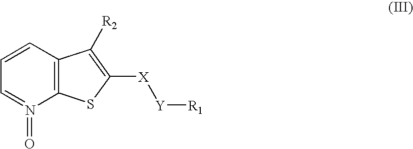
wherein the meaning of X, Y, R₁ and R₂ are as defined above for the formula (I); coupling the compound of formula (III) with trifluoroacetic anhydride or acetic anhydride in dimethylformamide to obtain a compound of formula (I); and, optionally thereafter forming one or more tautomers, salts, hydrates, or solvates of compounds of formula (I).

16. A process for the preparation of a compound of formula (I): 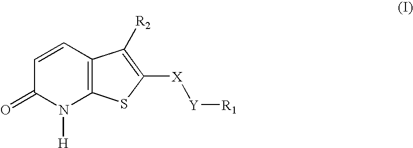
wherein:
X is selected from CO, SO, and SO₂;
Y is selected from O, OCH₂, (CH₂)n, NH, and NHCH₂;
n is an integer ranging from 0 to 2;
R₁ is an optionally substituted alkyl, cycloalkyl, phenyl, biphenyl, or heterocyclyl;
R₂ is an optionally substituted NR₃R₄ group, wherein R₃ and R₄ are independently selected from hydrogen and alkyl, or R₃ and R₄ together with the N atom to which they are attached form an optionally substituted C₅₋₇ heterocyclyl group containing one or more heteroatom(s) selected from N, O, and S; or tautomers, salts, hydrates, or solvates thereof, comprising: coupling the compound of formula (V): 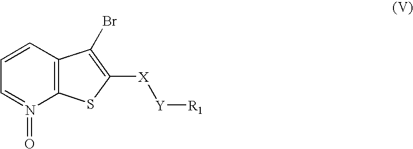
wherein the meaning of X, Y and R₁ are as defined above for the formula (I) with a compound of formula (VII): (VII)
HNR₃R₄ tm (VII)
wherein R₃ and R₄ are independently selected from hydrogen and alkyl, or R₃ and R₄ together with the N atom to which they are attached can from an optionally substituted C₅₋₇ heterocyclyl group containing one or more heteroatom(s) selected from N, O, and S, in dimethylformamide to obtain a compound of formula (III): 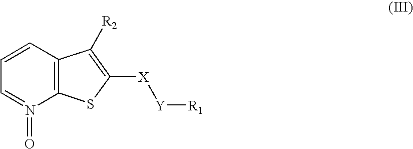
wherein the meaning of X, Y, R₁ and R₂ are as defined above for the formula (I); and reacting the compound of formula (III) with trifluoroacetic anhydride or acetic anhydride in dimethylformamide to obtain a compound of formula (I); and optionally thereafter forming one or more tautomers, salts, hydrates, or solvates of compounds of formula (I).

17. A pharmaceutical formulation comprising a therapeutically effective amount of a compound of formula (I): 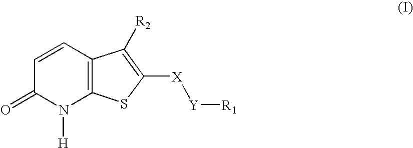
wherein:
X is selected from CO, SO, and SO₂;
Y is selected from O, OCH₂, (CH₂)ₙ, NH, and NHCH₂;
n is an integer ranging from 0 to 2;
R₁ is an optionally substituted alkyl, cycloalkyl, phenyl, biphenyl, or heterocyclyl;
R₂ is an optionally substituted phenyl heterocyclyl or NR₃R₄ group, wherein R₃ and R₄ are independently selected from hydrogen and alkyl, or R₃ and R₄ together with the N atom to which they are attached form an optionally substituted C₅₋₇ heterocyclyl group containing one or more heteroatom(s) selected from of N, O, and S; or tautomers, physiologically acceptable salts, hydrates or solvate thereof, and, one or more physiologically acceptable diluents, excipients and inert carriers.

18. A method of treating mGluR1 and mGluR5 receptor-mediated disorders, comprising: administering to a mammal in need of such treatment, a compound of formula (I) according to claim 13.

19. The method of claim 18, wherein said mGluR1 and mGluR5 receptor-mediated disorders are psychiatric disorders.

20. The method of claim 18, wherein said mGluR1 and mGluR5 receptor-mediated disorders are neurological disorders.

21. The method of claim 18, wherein said mGluR1 and GluR5 receptor-mediated disorders are chronic acute pain.

22. The method of claim 18, wherein said mGluR1 and mGluR5 receptor-mediated disorders are neuromuscular dysfunctions of the lower urinary tract or gastrointestinal disorders.

23. A method according to claim 18, wherein said mammal is a human.

24. A method of treating mGluR1 and mGluR5 receptor-mediated disorders, comprising: administering to a mammal in need of such treatment, a pharmaceutical formulation according claim 17.
